(19) United States
(12) Patent Application Publication
(10) Pub. No.: US 2002/0127249 A1
(43) Pub. Date: Sep. 12, 2002

(54) NOVEL FORMULATION FOR USE IN PAIN MANAGEMENT

(76) Inventors: BJORN BERGENSTAHL, LUND (SE); KATAYOUN WELIN-BERGER, SODERTALJE (SE)

Correspondence Address: PILLSBURY WINTHROP, LLP, P.O. BOX 10500, MCLEAN, VA, 22102, US

(21) Appl. No.: 09/214,016

(22) PCT Filed: Oct. 27, 1998
(86) PCT No.: PCT/SE98/01945

(30) Foreign Application Priority Data
Nov. 5, 1997 (SE) 9704031-5

(51) Int. Cl. A61K 9/00; A61K 6/00; A61K 7/00; A61K 31/135

(52) U.S. Cl. 424/400; 514/651

(57) ABSTRACT

The present invention is directed to a novel topical pharmaceutical water-in-oil composition in form of a cream or lotion, comprising

(i) 2-50% of a local anaesthetic in oil form in the final composition, or two or more local anaesthetics forming an oil when mixed together, effective to produce a topical anaesthetic effect upon administration;

(ii) 2-50% of a water-in-oil emulsifier, effective to produce an emulsion of the desired viscosity; and

(iii) 2-96% water.

Optionally the composition may contain up to 20% of pharmaceutically acceptable stabilizers or penetration enhancers.

This novel pharmaceutical composition is useful in therapy, in particularly for the treatment of pain.

### FIELD OF THE INVENTION

The present invention is directed to a new topical pharmaceutical water-in-oil composition, its use in therapy, particularly as a topical anaesthetic cream or lotion, as well as a process for preparing the pharmaceutical composition.

### BACKGROUND

Oil-in-water emulsions have by nature a low viscosity, and such compositions are not possible to use in form of a cream or lotion for topical application without the addition of also a thickening agent. Thus, in order to achieve a composition having a suitable viscosity for use as a topical cream or lotion, there is a need to add a thickening agent to such an oil-in-water emulsion. However, this provides the drawback with a retarded release rate of the active substance, which in turn means that the onset time of the active agent is increased.

Thus, the problem underlying the present invention is to provide a topical anaesthetic pharmaceutical water-in-oil composition in form of a cream or lotion, having sufficiently high viscosity/plasticity and a fast onset of action upon topical application.

### OUTLINE OF THE INVENTION

The problem identified above has now been solved by providing a new topical pharmaceutical water-in-oil composition in form of a cream or lotion, comprising the following ingredients:

(i) 2-50% of a local anaesthetic in oil form in the final composition, or two or more local anaesthetics forming an oil when mixed together, effective to produce a topical anaesthetic effect upon administration;

(ii) 2-50% of a water-in-oil emulsifier, effective to produce an emulsion of the desired viscosity; and

(iii) 2-96% water.

Optionally the above composition may contain up to 20% of pharmaceutically acceptable stabilizers or penetration enhancers.

The percentages are given as % by weight, based on the total weight of the composition.

When the ingredients are mixed together, to form a cream or lotion which is a water-in-oil formulation with the drug as the continous oil phase, the emulsion formed is a high internal phase emulsion with pronounced plasticity. Thus no external thickening agent is required in order to achieve the cream or lotion. The order of addition and type of mixing of ingredients as well as the choice of excipients determines the type of emulsion formed. The composition which is achieved, is a water-in-oil composition in the form of a cream or lotion, suitable for topical application to the skin.

The amount of local anaesthetic or mixture of local anaesthetics is preferably within the range from 5-20% by weight, based on the total weight of the composition.

Contrary to what is the case with an oil-in-water composition, it is possible to add also a thickening agent to the present reversed water-in-oil system, without conferring any negative effects on the release profile of the active agent.

Furthermore, it would be possible to add also an external oil to the pharmaceutical composition according to the present invention in an amount of up to a maximum of approximately 33% by weight in relation to the active ingredient, based on the total weight of the composition. However, it is preferred that the oil phase is provided to the pharmaceutical composition by being present in the active substance as such, or being formed when two or more substances are mixed together.

By local anaesthetic is intended substances providing anaesthesia after spinal administration or substances which have the capability to block a major nerve (Åkerman SBA, A Methodological study of spinal (subarachnoid) anaesthesia in the rat and mouse, British Journal of Anaesthesia, 1985, vol. 57, p. 943-948); (Shackell LF, tests of local anaesthetics by sciatic nerve block in the intact guinea pig, Anaesthesia and analgesia, 1935, January-February), and existing either alone or in combination with a second such substance in the form of an oil.

The local anaesthetics used in accordance with the present invention may be selected from any local anaesthetic which is present in oil form as such, or where an oil is formed when two or more local anaesthetics are melted together.

Examples of local anaesthetics suitable is the active agent in the pharmaceutical composition of the present invention are prilocaine, tetracaine, benzocaine, lidocaine, bupivacaine, and etidocaine, all being present in their free base forms.

Particularly preferred local anaesthetics as active agents are compounds of the general formula I

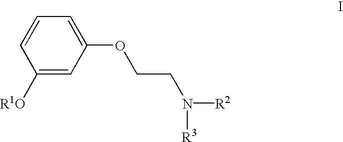

wherein

R¹ represents C₃₋₅ alkyl; and

R² and R³ independently represents C₁₋₃ alkyl;

provided that when R² and R³ both represent ethyl, then R¹ does not represent n-butyl, i-butyl or n-pentyl.

The preferred local anaesthetic according to the present invention is isopropyl-methyl-[2-(3-n-propoxy-phenoxy)-ethyl]-amine, which is a compound of the formula II

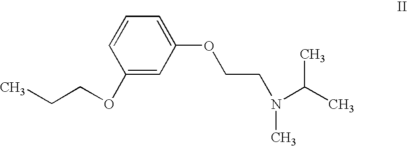

Further preferred local anaesthetic agents in accordance with the present invention is diisopropyl-[2-(3-n-propoxy-phenoxy)-ethyl]-amine, which is a compound of the formula

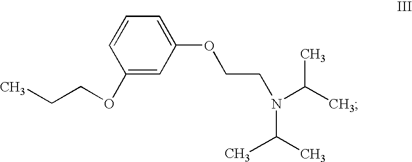

ethyl-isopropyl-[2-(3-n-propoxy-phenoxy)-ethyl]-amine, which is a compound of the formula

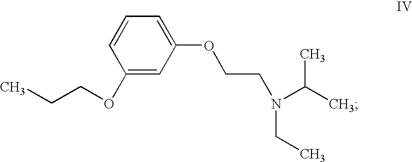

sameridine, which is a compound of the formula V

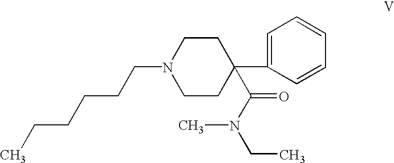

and a eutectic mixture of lidocaine and prilocaine (EMLA®).

Stabilizers or penetration enhancers may optionally be included in the composition according to the invention. Examples of stabilizers which may be used in the composition of the present invention are inorganic salts such as sodium chloride. Also pharmaceutically organic salts may be used.

Examples of penetration enhancers which may be used in the composition of the present invention are carbamide, alcohols etc. Specific examples of such penetration enhancers are urea, propylene glycol and ethanol. However, it will be appreciated by a person skilled in the art which penetration enhancers that would be suitable for the purpose.

By a water-in-oil emulsifier is intended emulsifiers which are oil-soluble and which are suitable for use in reversed systems. Suitable oil-soluble emulsifiers are disclosed in Bancraft W D, J. Physical Chemistry, Vol. 17, p. 501, 1913, which is hereby incorporated as reference.

Examples of water-in-oil emulsifiers suitable for use in the formulation of the present invention, are hydrofobic oil-soluble macromolecules, and hydrofobic low-molecular emulsifiers. The emulsifiers used in accordance with the present invention preferably have a HLB value of less than 8. For the definition of HLB value, reference is made to Davis J T, Proc. Intern. Congr. Surf. Activity, 2^{nd} ed., London 1957, p. 1426; Griffin W C, J. Soc. Cosmetic Chemists, p. 311-326, 1949. These examples should however not be construed as limiting the invention in any way.

Preferred water-in-oil emulsifiers according to the present invention are Polyglycerol esters, such as Grindsted PGPR90® (polyglyceryl-3-polyricinoleate), RYLO PG19® (polyglyceryl-3-polyricinoleate) (both from Danisco Ingredients AB), and Citrol® (from Croda); Polyethoxylated-7-hydrogenated ricinic oil, such as Cremophor WO7® (polyethoxylated-7-hydrogenated ricinic oil) (from BASF); Elfacos ST9® (Polyglycerol-45-dodecyl glycol copolymer) (Akzo Nobel); polysilaoxane, such as Abil EM90® (Cetyl Dimethicone Copolyol), Abil WE-09® (Polyglyceryl-4-isostearate, Cetyl Dimethicone Copolyol and Hexyl Laurate), and Abil WS 08® (all from Goldschmidt).

Preferably the amount of water-in-oil emulsifier is within the range from 5-15% by weight, based on the total weight of the composition.

A preferred amount of water in the water-in-oil composition of the present invention, is 70-95% by weight, based on the total weight of the composition.

By reversed system we mean a water-in-oil system, i.e. the reverse of an oil-in-water system.

The formulation is of medium to high viscosity dependent on the amount of water and/or type of emulsifier, and thus there is no need for addition of an external thickening agent in order to adjust the viscosity. However, addition of a thickening agent, if needed, will have no impact on the efficacy of the formulation, i.e. the onset time. As explained above, it is not possible to add a thickening agent to an oil-in-water formulation without increasing the onset time. Thus, a water-in-oil formulation according to the present invention, which is a reversed system, provides a great advantage compared to a normal phase emulsion. Addition of a thickening agent to an oil-in-water emulsion (normal phase emulsion) affects the release rate of the substance, and thus, the onset time will increase. This effect will be even more clear if the active ingredients of low water solubility. In the system according to the present invention, the high viscosity is generated through the structure developed by the presence of the emulsion droplets. Hence, there is a possibility to obtain a high viscosity with very limited effect on the release rate.

By the expression “fast onset of action” is intended that a local anaesthetic effect should be present in the subject in need of pain relief within preferably 30 minutes from the time for the topical application of the cream or emulsion comprising the active agent. However, the choice of active agent will determine the onset time. It should also be emphasized, that the high viscosity in the water-in-oil composition according to the present invention does not influence the onset time for the active substance, which as mentioned above, is contrary to what is the case in a normal system, i.e. an oil-in-water system.

The expression “topical application” will be appreciated by a person skilled in the art, and include application to the skin with or without occlusion.

As mentioned above, the composition according to the present invention is a water-in-oil emulsion. This allows a good contact between the active substance and the application site, because the substance constitutes the external phase of the formulation. This in turn provides a higher accessability of the active substance compared to what is the case for a normal oil-in-water emulsion of the same concentration. The advantage with a water-in-oil emulsion is that it has an occlusive effect by hydrating the upper layers of the stratum corneum and thereby inhibiting evaporation of eccrine sweet secretions. Thus, a further advantage with the composition according to the present invention, is that it may be used without an additional occlusive dressing.

No separate oil needs to be added to the composition of the present invention, since the oil is already present by the active component(s) as such. In the final composition, a fraction of the local anaesthetic or mixture of local anaesthetics are present in oil form. The size of this fraction, i.e. the amount of active ingredient present in the oil form, depends on the pH of the composition.

The pharmaceutical composition according to the present invention is intended to provide local anaesthesia by means of topical application on skin. The expression “skin” is intended to include mucous membranes, as well as intact and wounded skin.

A further aspect of the present invention is a pharmaceutical composition for use in therapy, in particularly for use as a local anaesthetic composition for pain management.

A further aspect of the present invention is a method for the treatment of pain, whereby a pharmaceutical composition according to the present invention is administered to a subject in need of pain relief.

The expression “pain” is defined as an unpleasant sensory and emotional experience associated with actual or potential tissue damage, or described in terms of such damage (International Association for the study of Pain, IASP, 1994).

Methods of Preparation

The pharmaceutical composition according to the present invention may be prepared by traditional emulsification techniques, see e.g. Becher P, Emulsions, Theory and practice, 2^{nd} edition., Reinhold publishing corporation, New York, USA, 1966, which is hereby incorporated as reference.

In order to prepare a water-in-oil composition according to the present invention, the ingredients are mixed as follows.

(i) The active agent, which is an oil as such, or the active agents in base form as such and which form an oil upon mixture, and the emulsifier, are weighed in proper amounts and mixed to total homogenity at room temperature or optionally under heating;

(ii) If salts, enhancers or any other additional ingredients are to be included, these are dissolved in water;

(iii) the water phase is thereafter slowly added to the oil phase while mixing at room temperature or optionally under heating providing a water-in-oil composition which is a cream or lotion.

### DETAILED DESCRIPTION OF THE INVENTION

The invention will now be described in sore detail by the following examples, which are not to be considered as limiting the invention.

In the specific Examples 1-14 given below, the compound isopropyl-methyl-[2-(3-n-propoxy-phenoxy)-ethyl]-amine was used as the active ingredient.

| | | [% by weight] |
|---|---|---|
| | Example 1 | |
| (i) | isopropyl-methyl-[2-(3-n-propoxy-phenoxy)- | 15 |
| | ethyl]-amine | |
| (ii) | Grindsted PGPR 90 ® | 10 |
| (iii) | H₂O | 74% |
| (iv) | Sodium chloride | 1% |
| | Example 2 | |
| (i) | isopropyl-methyl-[2-(3-n-propoxy-phenoxy)- | 15 |
| | ethyl]-amine | |
| (ii) | Rylo PG19 ® | 10 |
| (iii) | H₂O | 74 |
| (iv) | Sodium chloride | 1% |
| | Example 3 | |
| (i) | isopropyl-methyl-[2-(3-n-propoxy-phenoxy)- | 15 |
| | ethyl]-amine | |
| (ii) | Rylo PG19 ® | 10 |
| (iii) | H₂O | 75 |
| | Example 4 | |
| (i) | isopropyl-methyl-[2-(3-n-propoxy-phenoxy)- | 10 |
| | ethyl]-amine | |
| (ii) | Rylo PG19 ® | 6.7 |
| (iii) | H₂O | 83.3 |
| | Example 5 | |
| (i) | isopropyl-methyl-[2-(3-n-propoxy-phenoxy)- | 15 |
| | ethyl]-amine | |
| (ii) | Cremophor WO7 ® | 10 |
| (iii) | H₂O | 74 |
| (iv) | Sodium chloride | 1% |
| | Example 6 | |
| (i) | isopropyl-methyl-[2-(3-n-propoxy-phenoxy)- | 15 |
| | ethyl]-amine | |
| (ii) | Cremophor WO7 ® | 10 |
| (iii) | H₂O | 75 |
| | Example 7 | |
| (i) | isopropyl-methyl-[2-(3-n-propoxy-phenoxy)- | 10 |
| | ethyl]-amine | |
| (ii) | Cremophor WO7 ® | 6.7 |
| (iii) | H₂O | 83.3 |
| | Example 8 | |
| (i) | isopropyl-methyl-[2-(3-n-propoxy-phenoxy)- | 7.5 |
| | ethyl]-amine | |
| (ii) | Cremophor WO7 ® | 5.0 |
| (iii) | H₂O | 87.5 |
| | Example 9 | |
| (i) | isopropyl-methyl-[2-(3-n-propoxy-phenoxy)- | 15 |
| | ethyl]-amine | |
| (ii) | Abil EM 90 ® | 10 |
| (iii) | H₂O | 74 |
| (iv) | Sodium chloride | 1% |
| | Example 10 | |
| (i) | isopropyl-methyl-[2-(3-n-propoxy-phenoxy)- | 15 |
| | ethyl]-amine | |
| (ii) | Abil EM 90 ® | 10 |
| (iii) | H₂O | 75 |
| | Example 11 | |
| (i) | isopropyl-methyl-[2-(3-n-propoxy-phenoxy)- | 10 |
| | ethyl]-amine | |
| (ii) | Abil EM 90 ® | 6.7 |
| (iii) | H₂O | 83.3 |
| | Example 12 | |
| (i) | isopropyl-methyl-[2-(3-n-propoxy-phenoxy)- | 7.5 |
| | ethyl]-amine | |
| (ii) | Abil EM 90 ® | 5.0 |
| (iii) | H₂O | 87.5 |
| | Example 13 | |
| (i) | isopropyl-methyl-[2-(3-n-propoxy-phenoxy)- | 15 |
| | ethyl]-amine | |
| (ii) | Abil WE 09 ® | 10 |
| (iii) | H₂O | 74 |
| (iv) | Sodium chloride | 1 |
| | Example 14 | |
| (i) | isopropyl-methyl-[2-(3-n-propoxy-phenoxy)- | 15 |
| | ethyl]-amine | |
| (ii) | Elfacos ST9 ® | 10 |
| (iii) | H₂O | 74 |
| (iv) | Sodium chloride | 1 |

In the specific Example 15 given below, the compound diisopropyl-methyl-[2-(3-n-propoxy-phenoxy)-ethyl]-amine was used as the active ingredient.

| | Example 15 | [% by weight] |
|---|---|---|
| (i) | diisopropyl-[2-(3-n-propoxy-phenoxy)-ethyl]- | 8 |
| | amine | |
| (ii) | Abil EM 90 ® | 10 |
| (iii) | H₂O | 81 |
| (iv) | Sodium chloride | 1 |

In the specific Example 16 given below, the compound ethyl-isopropyl-[2-(3-n-propoxy-phenoxy)-ethyl]-amine was used as the active ingredient.

| | Example 16 | [% by weight] |
|---|---|---|
| (i) | ethyl-isopropyl-[2-(3-n-propoxy-phenoxy)- | 8 |
| | ethyl]-amine | |
| (ii) | Abil EM 90 ® | 10 |
| (iii) | H₂O | 81 |
| (iv) | Sodium chloride | 1 |

In the specific Example 17 given below, the compound sameridine was used as the active ingredient.

| | Example 17 | [% by weight] |
|---|---|---|
| (i) | sameridine | 8 |
| (ii) | Abil EM 90 ® | 10 |
| (iii) | H₂O | 81 |
| (iv) | Sodium chloride | 1 |

In the specific Example 18 given below, a eutectic mixture of lidocaine and prilocaine was used as the active ingredient.

| | Example 18 | [% by weight] |
|---|---|---|
| (i) | eutectic mixture of lidocaine and prilocaine | 10 |
| | (50:50 ratio) | |
| (ii) | Abil EM 90 ® | 12 |
| (iii) | H₂O | 77 |
| (iv) | Sodium chloride | 1 |

Biological Studies

The pharmaceutical compositions according to the present invention were tested according to the following In vivo method, TOPICAL ANAESTHESIA DURING OCCLUSION OF INTACT SKIN IN THE GUINEA-PIG, which is a modification of the method originally described by Edith Bülbring and Isabella Wajda in J Pharmacol Exp Ther 1945: 85: 78-84.

The hair is removed from the back of the guinea-pig with a depilatory cream (OPILCA® Hans Schwarzkopf GmbH, Hamburg, Germany). The hairless and smooth skin is washed with soap and water and the animal is kept in a cage under a desk lamp about two hours before experimentation. On pricking the back of the animal with a cannula (22G) KIFA (with no point) or a von Frey filament (4.74) (SEMMES-WEINSTEIN PRESSURE AESTHESIOMETER), a twitching of the skin is elicited. A circular piece of gauze (one up to eight layers) saturated with test formulation in a thin plastic cap (4.5 cm²) is applied to the middle of the back. The cup is then covered with Self-adhesive (FIXOMULL® BDF Beiersdorf AG Hamburg, Germany) and the occlusion is finally protected with an elastic bandage. At the end of the application period the treated area is wiped with a tissue and than examined for signs of local irritation. The skin which has been in contact with the formulation is pricked with the cannula or the von Frey filament under constant pressure six times at different places and the presence or absence of the twitching response in the skin of the treated area is noted. This procedure is repeated at regular intervals of five, ten or fifteen minutes. The first measured scores are recorded five minutes after the end of the application period.

The number of pricks not eliciting a response gives an indication of the degree of sensory anaesthesia or analgesia. Groups of three or six animals are used for each test formulation.

Permeation Experiment

Frozen stored (−20° C.) human skin, obtained from female donors after cosmetic surgery, was dermatomed to 0.25 mm (Padgett Electro Dermatome model B, Kansas City, Mo., USA). The skin was mounted in Franz-type permeation cells with the dermal side in direct contact with phosphate-buffered saline (PBS). The permeation cells had a diffusion area between 0.92 and 1.05 cm² and a receiver chamber volume between 13.00 and 16.04 ml. After overnight incubation at 4° C. the PBS was replaced with degassed PBS. The cells, kept at 32° C., were connected via the sampling port to a spectrophotometer (Lambda 20, Perkin-Elmer, Stockholm, Sweden) equipped with flowcells for on-line analysis of the receiver. The donor formulations were applied to the stratum corneum side of the skin. The receiver concentrations were re-calculated to flux values and plotted as function of time.

1. A topical pharmaceutical water-in-oil composition in form of a cream or lotion, comprising:
(i) 2-50% of a local anaesthetic in oil form in the final composition, or two or more local anaesthetics forming an oil when mixed together, effective to produce a topical anaesthetic effect upon administration;
(ii) 2-50% of a water-in-oil emulsifier, effective to produce an emulsion of the desired viscosity; and
(iii) 2-96% water;
the percentages being given as % by weight, based on the total weight of the composition.

2. A topical pharmaceutical water-in-oil composition according to claim 1, further comprising up to 20% of pharmaceutically acceptable stabilizers or penetration enhancers.

3. A topical pharmaceutical water-in-oil composition according to claim 1 or 2, comprising
(i) 5-20% of a local anaesthetic in oil form in the final composition, or two or more local anaesthetics forming an oil when mixed together, effective to produce a topical anaesthetic effect upon administration;
(ii) 5-15% of a water-in-oil emulsifier, effective to produce an emulsion of the desired viscosity; and
(iii) 70-95% water.

4. A topical pharmaceutical water-in-oil composition according to anyone of the previous claims, wherein the local anaesthetic is selected from one or more of prilocaine, tetracaine, benzocaine, lidocaine, bupivacaine, and etidocaine.

5. A topical pharmaceutical water-in-oil composition according to anyone of claims 1-3, wherein the local anaesthetic is a compound of the general formula I 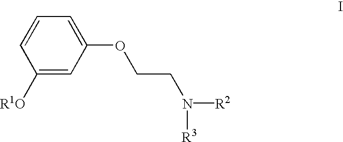
wherein
R¹ represents C₃₋₅ alkyl; and
R² and R³ independently represents C₁₋₃ alkyl;
provided that when R² and R³ both represent ethyl, then R¹ does not represent n-butyl, i-butyl or n-pentyl.

6. A topical pharmaceutical water-in-oil composition according to claim 5, wherein the local anaesthetic is isopropyl-methyl-[2-(3-n-propoxy-phenoxy)-ethyl]-amine.

7. A topical pharmaceutical water-in-oil composition according to claim 5, wherein the local anaesthetic is diisopropyl-[2-(3-n-propoxy-phenoxy)-ethyl]-amine.

8. A topical pharmaceutical water-in-oil composition according to claim 5, wherein the local anaesthetic is ethyl-isopropyl-[2-(3-n-propoxy-phenoxy)-ethyl]-amine.

9. A topical pharmaceutical water-in-oil composition according to anyone of claims 1-3, wherein the local anaesthetic is sameridine.

10. A topical pharmaceutical water-in-oil composition according to any of claims 1-4, wherein the active agent is a eutectic mixture of two or more local anaesthetics.

11. A topical pharmaceutical water-in-oil composition according to claim 10, wherein the active agent is a eutectic mixture of lidocaine and prilocaine.

12. A topical pharmaceutical water-in-oil composition according to any of the preceding claims, further comprising an inorganic salt.

13. A topical pharmaceutical water-in-oil composition according to claim 12, wherein the inorganic salt is sodium chloride.

14. A topical pharmaceutical water-in-oil composition according to any of the preceding claims, wherein the water-in-oil emulsifier is selected from hydrofobic oil-soluble macromolecules and hydrofobic low-molecular emulsifiers.

15. A topical pharmaceutical water-in-oil composition according to claim 14, wherein the water-in-oil emulsifier has a HLB value of less than 8.

16. A topical pharmaceutical water-in-oil composition according to any of the preceding claims, wherein the water-in-oil emulsifier is selected from anyone of polyglycerol esters, polyethoxylated-7-hydrogenated ricinic oil, and polysilaoxanes.

17. A topical pharmaceutical water-in-oil composition according to claim 16, wherein the water-in-oil emulsifier is selected from anyone of PGPR90 ®, RYLO PG19®, Citrol®, Cremophor WO7®, Elfacos ST9® and Abil EM90®.

18. A topical pharmaceutical water-in-oil composition according to any of the preceding claims, for use in therapy.

19. A topical pharmaceutical water-in-oil composition according to claim 18, for use in pain management.

20. A method for the treatment of pain, whereby a topical pharmaceutical water-in-oil composition of any of the preceding claims is topically administered to a subject in need of pain relief.

21. A process for the preparation of a pharmaceutical composition of claim 1, whereby
i) the active agent and the emulsifier are weighed in proper amounts and mixed to total homogenity at room temperature or optionally under heating;
(ii) salts, enhancers or any other additional ingredients are dissolved in water;
(iii) the water phase is thereafter slowly added to the oil phase while mixing at room temperature or optionally under heating, providing a water-in-oil composition which is cream or lotion.
